(19) United States
(12) Patent Application Publication
(10) Pub. No.: US 2017/0202501 A1
(43) Pub. Date: Jul. 20, 2017

(54) DRIVING ASSISTANCE SYSTEM

(71) Applicant: MAZDA MOTOR CORPORATION, Hiroshima (JP)

(72) Inventors: Junichiro KUWAHARA, Hiroshima-shi (JP); Hitomi NAKAZATO, Hiroshima-shi (JP)

(73) Assignee: MAZDA MOTOR CORPORATION, Hiroshima (JP)

(21) Appl. No.: 15/398,214

(22) Filed: Jan. 4, 2017

(30) Foreign Application Priority Data
Jan. 14, 2016 (JP) 2016-004957

(51) Int. Cl. A61B 5/18; A61B 5/00; G09B 9/04; G09B 19/00; B60W 40/08; G08G 1/01

(52) U.S. Cl. CPC A61B 5/18; B60W 40/08; G08G 1/0133; G09B 9/04; G09B 19/00; A61B 5/486; A61B 5/0077; A61B 5/6893; A61B 5/7264; B60R 2011/0003

(57) ABSTRACT

When finding the driver tensed by his or her increased external focus on driving, this system provides driving assistance that enhances his or her motivation to drive by encouraging him or her to drive actively and increase his or her internal focus on driving. The driving assistance may be provided when he or she has high driving skills, when there are no traffic jams, or when he or she is in good health. Examples of specific driving assistance include giving exemplary driving instructions to the driver, providing him or her with navigation to a road with features that would entertain him or her through driving, and improving sensitivity to any change in the vehicle's state responsive to driving operations.

### CROSS-REFERENCE TO RELATED APPLICATION

This application claims priority to Japanese Patent Application No. 2016-004957 filed on Jan. 14, 2016, the entire disclosure of which is hereby incorporated by reference.

### BACKGROUND

The present disclosure relates to a driving assistance system.

Recently, an increasing number of vehicles are equipped with various driving assistance capabilities, examples of which include automatic lane keeping control, automatic braking control, and adaptive auto-cruise control that have already been installed in a lot of vehicles on the market. For example, Japanese Unexamined Patent Publication No. H06-255519 discloses a driving assistance technique for relaxing the driver's attention to the surrounding environment in order to relieve his or her tension. Japanese Unexamined Patent Publication No. H07-069233 discloses a driving assistance technique for alleviating the driver's muscle tone during driving with the intervention of a control program.

Some external factors may oblige the driver to drive his or her vehicle passively (i.e., against his or her will), thus impelling him or her to increase his or her external focus on driving and ultimately making him or her feel tension rising. Such tension is not beneficial because it makes the driver feel that he or she is doing “forced driving.” Thus, the tension should be alleviated as much as possible. In contrast, if the driver feels he or she is driving his or her vehicle actively (i.e., comfortably at his or her own will), then he or she is certainly compelled to increase his or her internal focus on driving but does not feel such tension rising.

### SUMMARY

The present disclosure provides a driving assistance system for alleviating the tension of a driver who is impelled to drive his or her vehicle passively and increase his or her external focus on driving.

Specifically, a driving assistance system according to the present disclosure includes:

a tension gauging unit configured to determine whether or not a driver is tensed by being forced to drive his or her vehicle passively and highly increase his or her external focus on driving; and

a driving assistance unit configured to provide, if the tension gauging unit has found the driver tensed, driving assistance that enhances the driver's motivation to drive by encouraging him or her to drive actively and thereby increase his or her internal focus on driving.

This configuration alleviates the driver's tension, resulting from his or her increased external focus on driving, by providing such driving assistance for the driver as to allow him or her to increase his or her internal focus on driving. In particular, since the driving assistance is provided to increase the driver's internal focus, this enhances his or her motivation to drive and improves his or her driving skills beneficially.

This basic idea of the present disclosure may be implemented as the following exemplary embodiments:

In one exemplary embodiment, the driving assistance system further includes:

a driver's condition detecting unit configured to detect the driver's condition;

a vehicle operating state detecting unit configured to detect the driver's vehicle operating state; and

an equipment operating state detecting unit configured to detect the driver's operating state of onboard equipment irrelevant to his or her vehicle driving operations.

The system determines, based on results of detection obtained by the respective detecting units, the driver's degree of focus on driving and his or her degree of leeway in driving.

The system finds the driver tensed if his or her degree of focus on driving is high, his or her degree of leeway in driving is low, and his or her external focus on driving has been increased by some external factors.

According to this embodiment, the driver's condition may be determined based on his or her degree of focus on driving and his or her degree of leeway in driving, and a determination may be made highly accurately, based on this decision, that he or she is tensed due to a significant increase in his or her external focus on driving. This alleviates his or her tension effectively through the driving assistance.

In another exemplary embodiment, the driving assistance system further includes

a driving skills assessment unit configured to assess the driver's driving skills.

If the driving skills assessment unit has determined his or her driving skills to be high, the driving assistance unit provides the driving assistance that enhances the driver's motivation to drive by encouraging him or her to increase his or her internal focus on driving.

According to this embodiment, the driving assistance is provided if the driver's driving skills have turned out to be high. This further enhances his or her motivation to drive and further improves his or her driving skills highly beneficially.

In still another exemplary embodiment, the driving assistance system further includes:

a physical condition detecting unit configured to detect the driver's physical condition; and

a traffic jam detecting unit configured to detect a traffic jam.

If the physical condition detecting unit has found the driver in good health and the traffic jam detecting unit has detected no traffic jams, the driving assistance unit provides the driving assistance that enhances the driver's motivation to drive by encouraging him or her to increase his or her internal focus on driving.

According to this embodiment, the system provides driving assistance after having confirmed that this is a situation in which the driver would be motivated to drive. This efficiently alleviates his or her tension and improves his or her driving skills involved with his or her enhanced motivation to drive.

In yet another exemplary embodiment, the driving assistance system further includes:

a driver's condition detecting unit configured to detect the driver's condition;

a vehicle operating state detecting unit configured to detect the driver's vehicle operating state;

an equipment operating state detecting unit configured to detect the driver's operating state of onboard equipment irrelevant to his or her vehicle driving operations; and

a classification unit configured to classify, based on results of detection obtained by the respective detecting units, the driver's condition as any of a plurality of types using, as parameters, his or her degree of focus on driving and his or her degree of leeway in driving.

The classification unit classifies the driver's condition as:

a first type if his or her degree of focus on driving is low, his or her degree of leeway in driving is low, and his or her degree of distraction from driving is high;

a second type if his or her degree of focus on driving is low and his or her degree of leeway in driving is high;

a third type if his or her degree of focus on driving is high, his or her degree of leeway in driving is low, and his or her degree of external focus on driving has been increased by some external factors; or

a fourth type if his or her degree of focus on driving is high, his or her degree of leeway in driving is high, and his or her degree of internal focus on driving is high.

If the classification unit classifies the driver's condition as the third type, the tension gauging unit finds the driver tensed.

According to this embodiment, the driver's condition is classified as any of these four types by using, as parameters, his or her degree of focus on driving and his or her degree of leeway in driving, thereby highly accurately determining that the driver is tensed due to a significant increase in his or her external focus on driving. This alleviates his or her tension highly effectively through the driving assistance.

In yet another exemplary embodiment, the driving assistance system further includes:

a degree of internal focus measuring unit configured to measure the degree of internal focus of the driver who is driving actively;

a degree of external focus measuring unit configured to measure the degree of external focus of the driver who is driving passively;

a degree of distraction measuring unit configured to measure the driver's degree of distraction from driving operations; and

a degree of spare capacity measuring unit configured to measure the degree of spare capacity indicating the driver's degree of loss of focus.

The classification unit classifies, based on results of detection obtained by these four measuring units, the driver's condition as any of the various types.

According to this embodiment, the driver's condition may be classified highly accurately as any of the four types by measuring the respective degrees of focus, the degree of distraction and the degree of spare capacity. This alleviates his or her tension highly effectively.

In yet another exemplary embodiment, the classification unit classifies the driver's condition as any of the various types by regarding the degree of internal focus and the degree of spare capacity as values indicating the driver's degree of leeway and also regarding the degrees of internal and external foci as values indicating the driver's degrees of focus.

According to this embodiment, the driver's condition may be classified highly accurately as any of the four types by appropriately setting the degrees of focus on driving and the degree of leeway.

In yet another exemplary embodiment, the driver's condition detecting unit includes an image capturing unit configured to capture an image covering at least the driver's face, and the vehicle operating state detecting unit includes at least two sensors configured to detect respective operating states of an accelerator pedal and a brake pedal.

According to this embodiment, the respective degrees of focus, the degree of distraction, and the degree of spare capacity may be measured highly accurately by means of general electronic devices.

In yet another exemplary embodiment, the driving assistance includes giving exemplary driving instructions to the driver.

This sufficiently enhances the driver's motivation to drive, effectively alleviates his or her tension, and significantly improves his or her driving skills.

In yet another exemplary embodiment, the driving assistance includes providing the driver with navigation to a road with features that would entertain him or her through driving.

This sufficiently enhances the driver's motivation to drive, effectively alleviates his or her tension, and significantly improves his or her driving skills.

In yet another exemplary embodiment, the driving assistance includes improving sensitivity to any change in the vehicle's state responsive to driving operations.

This sufficiently enhances the driver's motivation to drive, effectively alleviates his or her tension, and significantly improves his or her driving skills.

### BRIEF DESCRIPTION OF THE DRAWINGS

FIG. 1 is a block diagram illustrating an exemplary control system according to an embodiment.

FIG. 2 is a table summarizing the details of four different types of driver's condition.

FIG. 3 is a table summarizing how the driver's condition is determined to be one of the four different types based on various parameters.

FIG. 4 is a flowchart illustrating an exemplary procedure of control according to an embodiment.

FIGS. 5-7 are a flowchart illustrating a detailed procedure of the driver's condition determination step Q1 shown in FIG. 4.

FIG. 8 is a flowchart illustrating an exemplary procedure of control for assessing the driver's driving skills according to an embodiment.

FIG. 9 is a table showing specific data used for assessing the driver's driving skills according to an embodiment.

FIG. 10 diagrammatically illustrates exemplary control for enhancing the driver's motivation to drive according to an embodiment.

FIG. 11 is a flowchart illustrating an exemplary procedure of control for storing the features of a road that entertained the driver according to an embodiment.

FIG. 12 diagrammatically illustrates how the throttle characteristics may be changed according to an embodiment.

FIG. 13 is a flowchart illustrating an exemplary procedure of control for lightening the driver's driving operation load according to an embodiment.

FIG. 14 is a flowchart illustrating an exemplary procedure of control for reducing the driver's cognitive level of external factors according to an embodiment.

### DETAILED DESCRIPTION

Exemplary embodiments will now be described in detail with reference to the accompanying drawings.

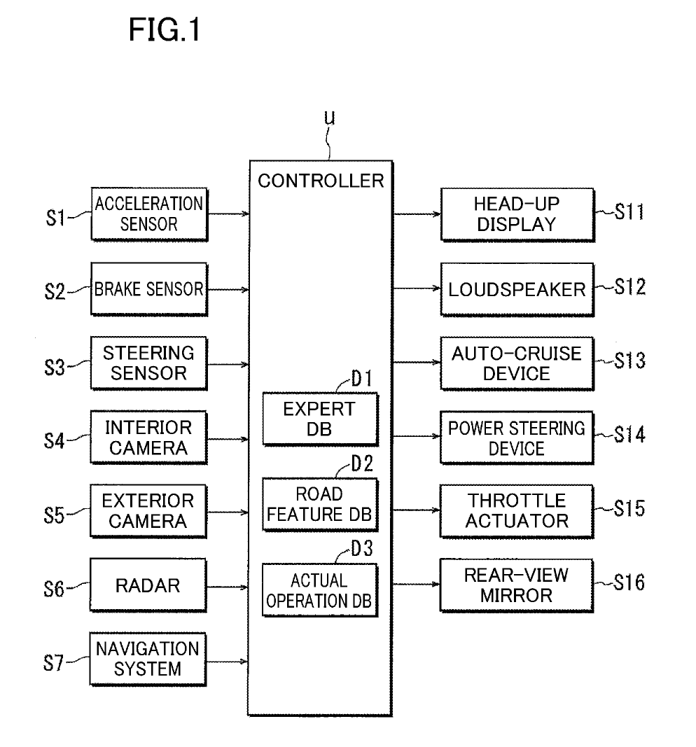

FIG. 1 illustrates an exemplary control system according to an embodiment. As shown in FIG. 1, a controller (control unit) U implemented as a microcomputer receives signals from various sensors and devices S1-S7 installed in a vehicle such as an automobile. Specifically, an acceleration sensor S1 is provided to detect the vehicle's accelerator position. A brake sensor S2 is provided to detect how far the vehicle's brake pedal is depressed (hereinafter referred to as a “degree of depression of the brake pedal”). A steering sensor S3 is provided to detect the steering angle. An interior camera S4 functioning as an image capture unit is provided to capture an image of the driver's face. An exterior camera S5 is provided to monitor the situation outside of the vehicle (e.g., the situation in front of his or her own vehicle, in particular). A radar S6 is provided to measure the distance to an obstacle in front of the vehicle, for example. A navigation system S7 is provided to get map information and information about the vehicle's current location.

The controller U controls the various devices S11-S16 for the purpose of providing driving assistance to be described later. Specifically, a head-up display S11 is provided in front of the driver's seat. A loudspeaker S12 is provided to emit sound inside the vehicle cabin. An adaptive auto-cruise device S13 is provided to be operable in the entire vehicle speed range (i.e., from a very low speed of slightly more than 0 km/h to the maximum speed) according to an embodiment. A power steering device S14 is provided to perform automatic steering, in particular. A throttle actuator S15 is provided to allow the driver to control the throttle characteristic (specifically, control the throttle opening with respect to the accelerator position). A rear-view mirror S16 is provided to allow the driver to adjust its reflectance using a liquid crystal display, for example, and thereby change the driver's view of the situation behind the vehicle (e.g., a view of the following vehicle).

To provide various types of driving assistance to be described later, the controller U includes three databases (DB) D1, D2, and D3. These databases D1-D3 are actually implemented as externally connected storage devices with large storage capacities. Specifically, the database D1 stores information about exemplary driving operations to be performed by an expert (i.e., experienced) driver (including data about the degrees of depression of the accelerator pedal, the degrees of depression of the brake pedal, and the timings to turn the steering wheel and degrees of the steering wheel turned) such that those exemplary driving operations are applicable to any of various traveling situations. The database D2 stores information about the features of a road that entertained the driver. The database D3 stores information about the driving operations actually performed by the driver (e.g., how the accelerator pedal and brake pedal have been pumped by the driver in an exemplary embodiment).

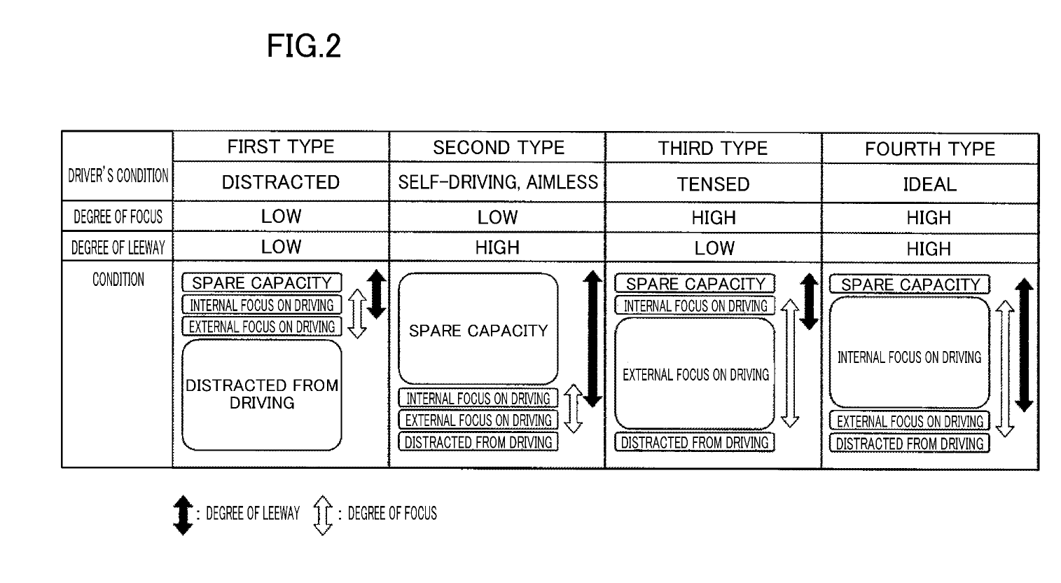

Next, it will be described with reference to FIG. 2 how to classify the driver's condition as any of four types. First of all, the driver's degrees of focus on driving and his or her degree of leeway in driving will be described as a premise of this type classification.

The focus on driving may be represented, depending on the driver's behavioral pattern, by any of the four different degrees, which will be hereinafter referred to as first, second, third, and fourth degrees, respectively, for convenience sake. Specifically, the first degree represents an external (passive) focus on driving, i.e., a situation where the driver is forced to perform driving operations against his or her will, and is less motivated to drive, due to some external factors. The second degree represents an internal (active) focus on driving, i.e., an ideal situation where the driver is driving his or her vehicle at his or her own will and is highly motivated to drive. The third degree represents the driver's distraction from driving (such as operating the touchscreen of the navigation system or talking over his or her cellphone). The fourth degree represents the driver's loss of focus (which is defined to be a redundancy that is an unused, reserved portion of his or her maximum focusing ability and will be hereinafter referred to as a “spare capacity” in the following description and drawings).

Supposing the sum of these four degrees of focus is 100%, the degree of focus on driving is the percentage accounted for by the sum of the external and internal foci, and the driver's degree of leeway in driving is the percentage accounted for by the sum of the internal focus and the spare capacity.

The following first, second, third, and fourth types are defined as the four types. Specifically, the driver's condition is classified as the first type if his or her degree of focus on driving is low and his or her degree of leeway in driving is low (particularly when the driver is doing distracted driving (e.g., looking aside while driving his or her vehicle)). The driver's condition is classified as the second type if his or her degree of focus on driving is low and his or her degree of leeway in driving is high (e.g., when his or her vehicle is self-driving or when he or she is doing aimless driving). The driver's condition is classified as the third type if his or her degree of focus on driving is high and his or her degree of leeway in driving is low. The driver in this third type of condition is found tensed, for example, when the following vehicle is running close behind his or her vehicle. The driver's condition is classified as the fourth type if his or her degree of focus on driving is high and his or her degree of leeway in driving is high. This is an ideal driving condition.

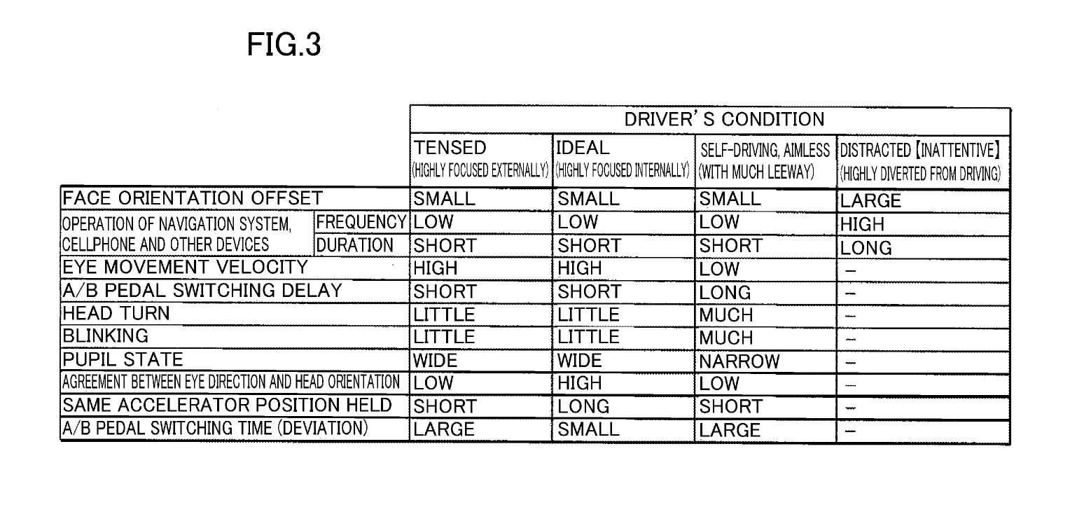

FIG. 3 is a table summarizing how the driver's condition, including the degrees of external and internal foci, the degree of distraction from driving, and the degree of spare capacity, is determined based on various parameters. Examples of such parameters include the driver's physical features extracted from the image captured by the interior camera S4 such as his or her facial expressions, eye direction, and pupil state and how the accelerator pedal (A pedal) and brake pedal (B pedal) are operated. The driver's current condition is determined to be any of the four types based on these results of detection integrated together.

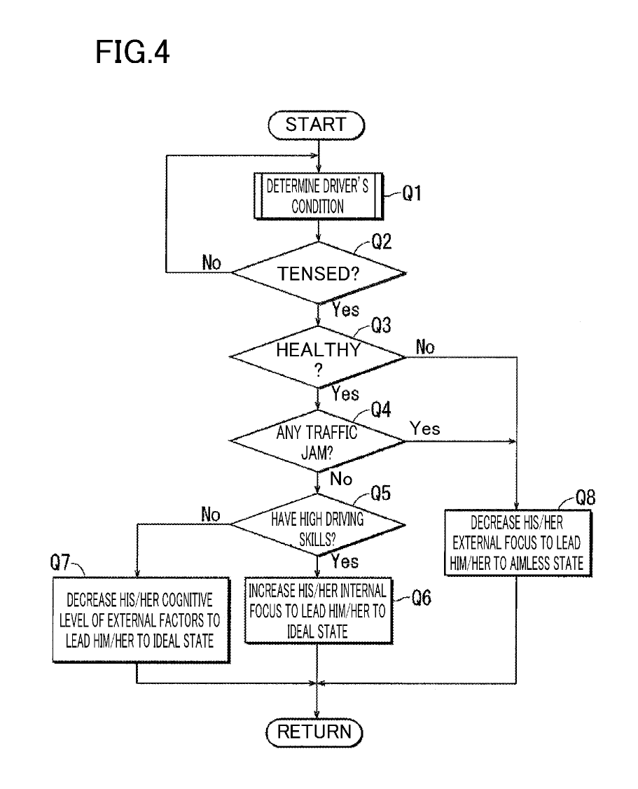

When determining the driver's condition to be the third type (i.e., when finding him or her tensed), the controller U performs a procedure of control to alleviate his or her degree of external focus through driving assistance. An exemplary procedure of control to be performed by the controller U will be described with reference to the flowcharts of FIGS. 4 through 7. In the following description, the reference sign Q denotes a processing step.

First of all, in Q1 shown in FIG. 4, the controller U determines the driver's condition as will be described later. In this processing step Q1, the controller U determines which of the four types the driver's current condition falls under. Next, in Q2, the controller U determines whether or not the driver is tensed, i.e., whether or not the driver's current condition falls under the third type. If the answer to the question of Q2 is NO, then no driving assistance is needed at this time, and the process goes back to Q1.

On the other hand, if the answer to the question of Q2 is YES, then the controller U determines in Q3 whether or not the driver's physical condition is good. As used herein, the driver's physical condition refers to not only some kind of malaise such as high fever but also drowsiness that prevents him or her from driving the vehicle properly. This determination may be made based on the information collected by the interior camera S4. Optionally, the determination may also be made with additional pieces of information about the driver's physical condition, including his or her heart rate (measured by a heart rate sensor provided for a seat cushion, for example), his or her skin resistance or skin temperature (measured by a resistance sensor or temperature sensor provided for the steering wheel, for example), and his or her body surface temperature (measured by an infrared sensor, for example), taken into account as well.

If the answer to the question of this processing step Q3 is YES, then the controller U determines in Q4 whether or not there is a traffic jam. This determination may be made based on the situation in front of the vehicle as monitored by the exterior camera S5, traffic jam information provided by the navigation system S7, and/or traffic jam information provided through communications with a service center, for example.

If the answer to the question of this processing step Q4 is NO (i.e., if there is no traffic jam), then the controller U determines in the next processing step Q5 whether or not the driver's driving skills are high. If the answer to the question of this processing step Q5 is YES, then the controller U provides driving assistance to increase his or her degree of internal focus on driving as will be described later and thereby decrease his or her degree of external focus on driving. On the other hand, if the answer to the question of this processing step Q5 is NO, then the controller U provides driving assistance to reduce the driver's cognitive level of external factors and thereby decrease his or her degree of external focus on driving.

If the answer to the question of Q3 is NO (i.e., if the driver is in bad health), then the process proceeds to Q8. Likewise, the process also proceeds to Q8 if the answer to the question of Q4 is YES (i.e., if there is a traffic jam). In Q8, the controller U provides driving assistance to lighten the driver's load of driving operations as will be described later and thereby decrease his or her degree of external focus on driving.

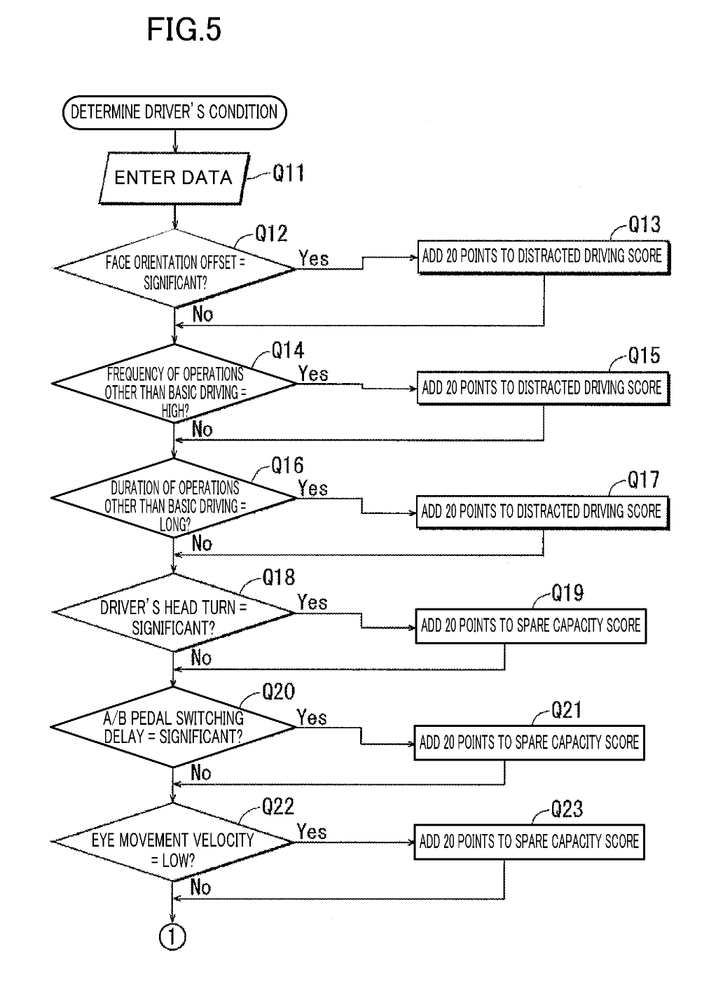

FIGS. 5-7 show the details of the processing step Q1 shown in FIG. 4. Specifically, after data has been entered in Q11 shown in FIG. 5, the controller U determines in Q12 whether or not the driver's face orientation has a significant offset (i.e., whether or not he or she is looking aside while driving). If the answer to the question of this processing step Q12 is YES, then a predetermined number of points (e.g., 20 points) are added to a distracted driving score in Q13.

After the controller U has performed the processing step Q13 or if the answer to the question of Q12 is NO, the controller U determines in Q14 whether or not the driver is frequently performing operations other than basic driving operations (i.e., pumping the accelerator and brake pedals, turning the steering wheel, and other driving operations to change the vehicle's behavior). More specifically, the controller U determines whether or not the frequency of occurrence of such non-basic operations is equal to or greater than a predetermined threshold value. If the answer to the question of this processing step Q14 is YES, then a predetermined number of points (e.g., 20 points) are added to the distracted driving score in Q15.

After the controller U has performed the processing step Q15 or if the answer to the question of Q14 is NO, the controller U determines in Q16 whether or not the duration of those other operations performed by the driver is significantly long compared to that of the basic driving operations. More specifically, the controller U determines whether or not the duration is equal to or greater than a predetermined threshold value. If the answer to the question of this processing step Q16 is YES, then a predetermined number of points (e.g., 20 points) are added to the distracted driving score in Q17.

After the controller U has performed the processing step Q17 or if the answer to the question of Q16 is NO, the controller U determines in Q18 whether or not the driver's head turn is significant. More specifically, the controller U determines whether or not his or her head turn is equal to or greater than a predetermined threshold value. If the answer to the question of this processing step Q18 is YES, then a predetermined number of points (e.g., 20 points) are added to a spare capacity score in Q19.

After the controller U has performed the processing step Q19 or if the answer to the question of Q18 is NO, the controller U determines in Q20 whether or not a significant delay has been caused by the driver in changing the pedals to pump from the accelerator pedal to the brake pedal, and vice versa. More specifically, the controller U determines whether or not the delay is equal to or greater than a predetermined threshold value. If the answer to the question of this processing step Q20 is YES, then a predetermined number of points (e.g., 20 points) are added to a spare capacity score in Q21.

After the controller U has performed the processing step Q21 or if the answer to the question of Q20 is NO, the controller U determines in Q22 whether or not the driver's eye movement velocity is low. More specifically, the controller U determines whether or not the eye movement velocity is equal to or smaller than a predetermined threshold value. If the answer to the question of this processing step Q22 is YES, then a predetermined number of points (e.g., 20 points) are added to a spare capacity score in Q23.

After the controller U has performed the processing step Q23 or if the answer to the question of Q22 is NO, the controller U determines in Q31 shown in FIG. 6 whether or not the degree of agreement between the driver's face orientation and his or her eye direction is high. More specifically, the controller U determines whether or not the degree of agreement falls within a predetermined threshold range. If the answer to the question of this processing step Q31 is YES, then a predetermined number of points (e.g., 20 points) are added to an actively (internally) focused driving score in Q32.

After the controller U has performed the processing step Q32 or if the answer to the question of Q31 is NO, the controller U determines in Q33 whether or not the same accelerator position has been held for a significant amount of time. More specifically, the controller U determines whether or not the amount of time is equal to or greater than a predetermined threshold value. If the answer to the question of this processing step Q33 is YES, then a predetermined number of points (e.g., 20 points) are added to an actively (internally) focused driving score in Q34.

After the controller U has performed the processing step Q34 or if the answer to the question of Q33 is NO, the controller U determines in Q35 whether or not the deviation (i.e., the standard deviation) in the amount of time it takes for the driver to change pedals to pump from the accelerator pedal to the brake pedal, and vice versa, is insignificant. More specifically, the controller U determines whether or not the standard deviation is equal to or smaller than a predetermined threshold value. If the answer to the question of this processing step Q35 is YES, then a predetermined number of points (e.g., 20 points) are added to an actively (internally) focused driving score in Q36.

If the answer to the question of Q35 is NO, then a predetermined number of points (e.g., 50 points) are added to a passively (externally) focused driving score in Q37.

After having performed the processing step Q37, the controller U determines in Q41 shown in FIG. 7 whether or not the (total) distracted driving score is greater than 0 points. If the answer to the question of this processing step Q41 is YES, then the controller U determines in Q42 that the driver is doing distracted driving (e.g., looking aside while driving), i.e., the driver's current condition falls under the first type.

On the other hand, if the answer to the question of the processing step Q41 is NO, then the controller U determines in Q43 whether or not the (total) spare capacity score is greater than the sum of the (total) passively focused driving score and the (total) actively focused driving score. If the answer to the question of this processing step Q43 is YES, then the controller U determines in Q44 that the driver is doing aimless driving, i.e., the driver's current condition falls under the second type.

On the other hand, if the answer to the question of the processing step Q43 is NO, then the controller U determines in Q45 whether or not the (total) actively focused driving score is greater than the (total) passively focused driving score. If the answer to the question of this processing step Q45 is YES, then the controller U determines in Q46 that the driver is in an ideal condition, i.e., the driver's current condition falls under the fourth type. On the other hand, if the answer to the question of this processing step Q45 is NO, then the controller U determines in Q47 that the driver is in a tense condition, i.e., the driver's current condition falls under the third type.

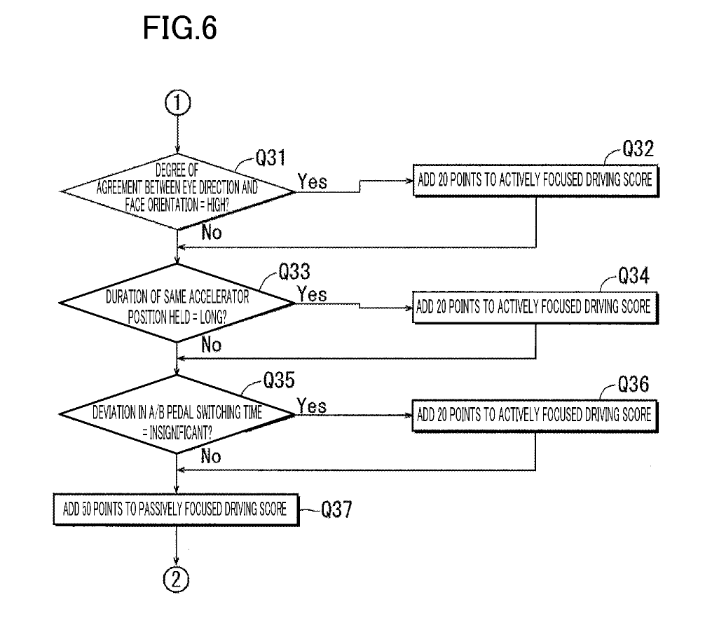

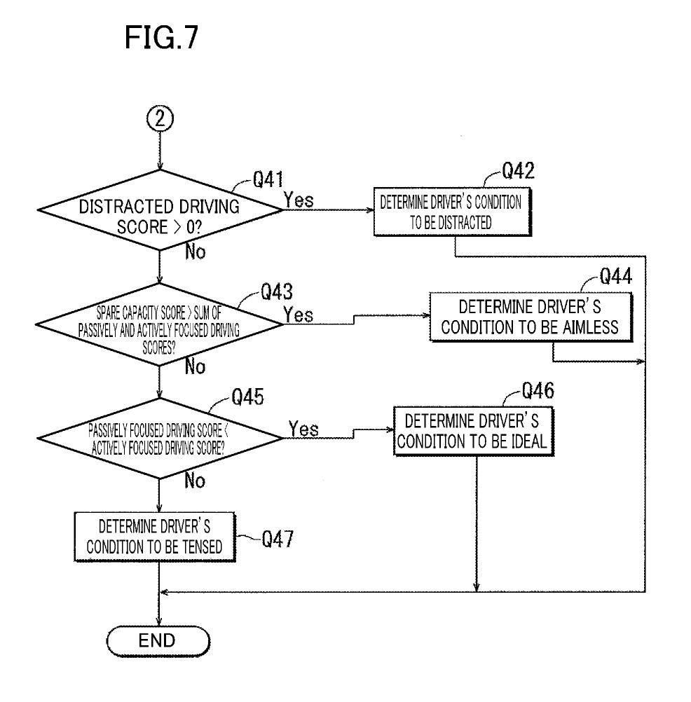

Next, a technique for assessing the driver's driving skills corresponding to the processing step Q5 shown in FIG. 4 will be described with reference to the flowchart of FIG. 8 and the table of FIG. 9. First of all, in Q51 shown in FIG. 8, the controller U issues an instruction to make a driving skills assessment (specifically, sets the amount of time it takes to get data for making a driving skills assessment). Next, in Q52, the controller U accumulates data about the actual operations of the accelerator and brake pedals for a predetermined amount of time (and stores it in the actual operation database D3). Thereafter, in Q53, the controller U calculates the driving skills score by comparing the data about the expert driver's operations retrieved from the database D1 to the data about the present driver's operations stored in the database D3. Subsequently, in Q54, the controller U determines, based on the driving skills score, whether the driver's driving skills are high or low.

In the processing step Q53, the driving skills score may be calculated as shown in FIG. 9, for example. Specifically, the points are added based on the time differences (which are set in three stages according to an exemplary embodiment) from the expert driver's performance in terms of the three parameters of: the amount of time for which the same accelerator position has been held; the amount of delay caused by the driver before he or she starts depressing the accelerator pedal; and the amount of time it has taken for the driver to change the pedals to pump from the accelerator pedal to the brake pedal, and vice versa. In Q54, the controller U determines the driver's driving skills to be high if the sum of the respective points with respect to these three parameters is equal to or greater than a predetermined threshold value, and to be low if the sum is less than the predetermined threshold value.

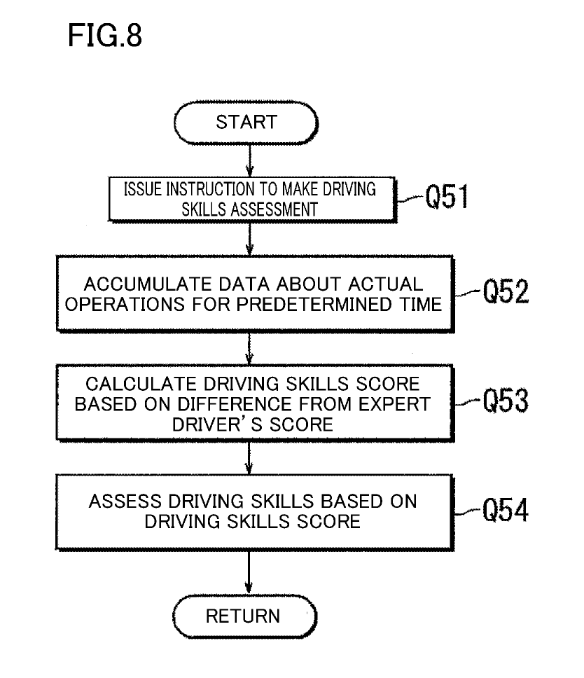

Next, an exemplary technique for increasing the driver's internal focus on driving in Q6 shown in FIG. 4 will be described with reference to FIGS. 10-12. Specifically, FIG. 10 illustrates an embodiment in which exemplary driving instructions are displayed on the screen to the driver. For example, while the driver is making cornering, not only an accelerating region and a decelerating region (in two different colors, for example) but also a steering point indicating a steering start timing (in a flickering state, for example) may be highlighted on the road image displayed. This enhances the driver's motivation to drive and improves his or her driving skills.

Although an exemplary cornering technique is illustrated in FIG. 10, any other appropriate set of exemplary driving instructions may also be displayed as an ideal example to follow, which may be a set of exemplary driving operations to perform on a highway or a set of exemplary parking operations, depending on the real-time situation on the road. Among other things, it is particularly beneficial to display a steering timing and timings to depress the accelerator and brake pedals as examples to follow. Optionally, the driver may be notified of these timings through audio guidance.

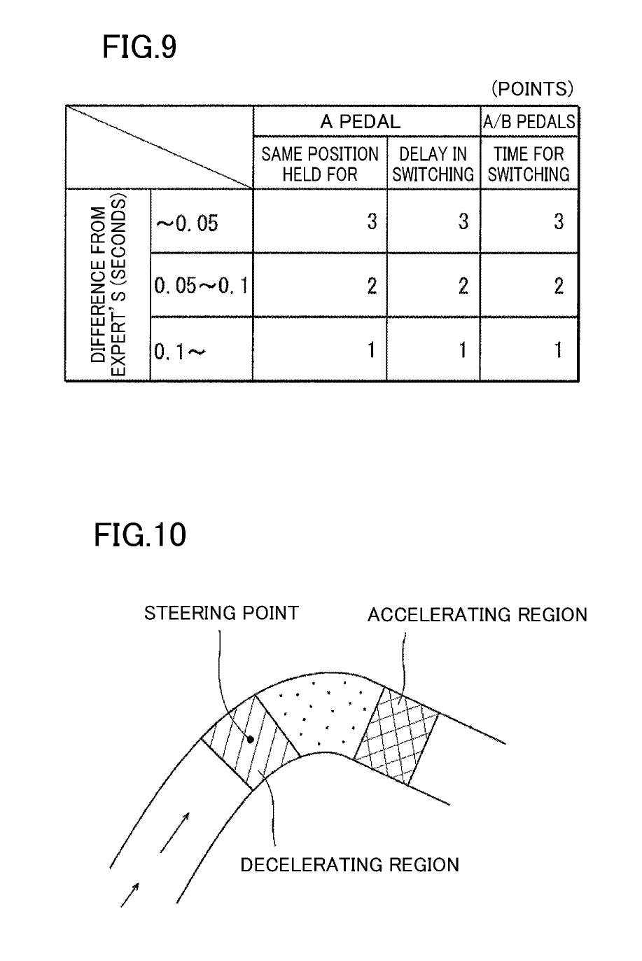

FIG. 11 shows an exemplary procedure of control to be performed to provide the driver with navigation to a road with features that would entertain him or her. Specifically, in Q61, the controller U estimates the driver's emotions based on his or her facial expressions captured by the interior camera S4, for example. Next, in Q62, the controller U determines, based on the results of estimation obtained in Q61, whether or not the driver is enjoying driving, i.e., entertained or amused by the features of the road. If the answer to the question of this processing step Q62 is YES, the features of the road entertaining him or her are stored in the database D2. On the other hand, if the answer to the question of the processing step Q62 is NO, then the process returns with the processing step Q63 skipped. Then, in the processing step Q6 shown in FIG. 4, the controller U selects, from among a plurality of roads running in almost the same direction as the road currently taken, a road having the features stored in the database D2, and provides the driver with navigation to this selected road (e.g., by providing guidance on a navigation screen image).

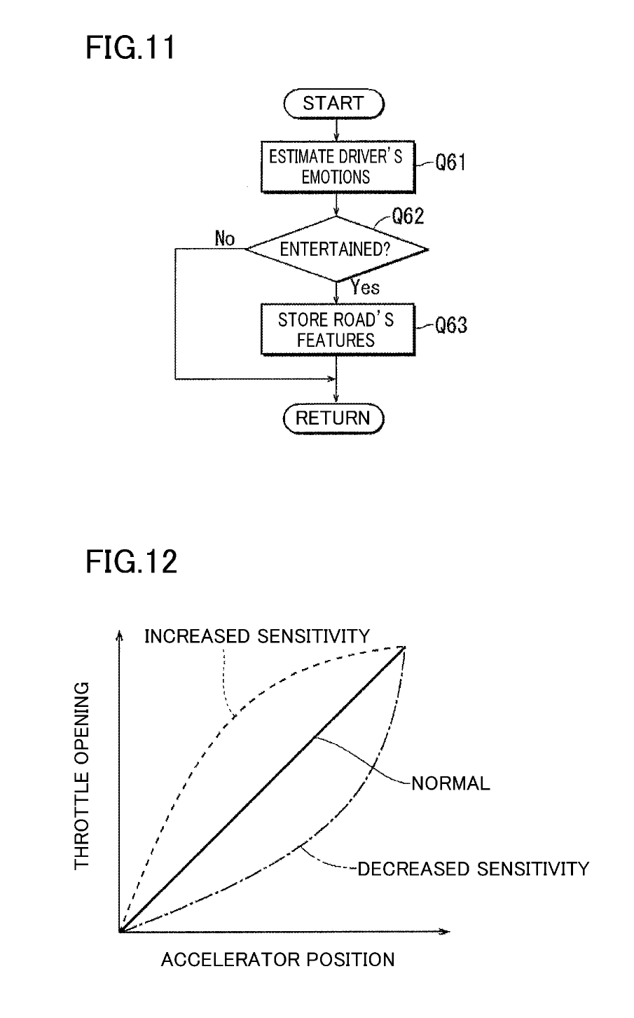

FIG. 12 illustrates an exemplary technique for enhancing the driver's motivation to drive by increasing the sensitivity of an engine output to accelerating operations. In FIG. 12, the solid line represents a normal (ordinary) throttle characteristic. The broken curve represents a throttle characteristic with increased sensitivity (i.e., a characteristic that the throttle opening increases more significantly with respect to the same accelerator position than in the normal throttle characteristic). The one-dot-chain curve represents a throttle characteristic with decreased sensitivity (i.e., a characteristic that the throttle opening decreases more significantly with respect to the same accelerator position than in the normal throttle characteristic). In Q6 shown in FIG. 4, the throttle characteristic with the increased sensitivity as indicated by the broken curve in FIG. 12 is selected, and the throttle actuator S15 is controlled in accordance with this selected throttle characteristic. Optionally, any two or all three of the control procedures shown in FIGS. 10-12 may be performed. Naturally, the driving assistance for increasing the driver's internal focus on driving does not have to be as described above, but may also be any other appropriate one.

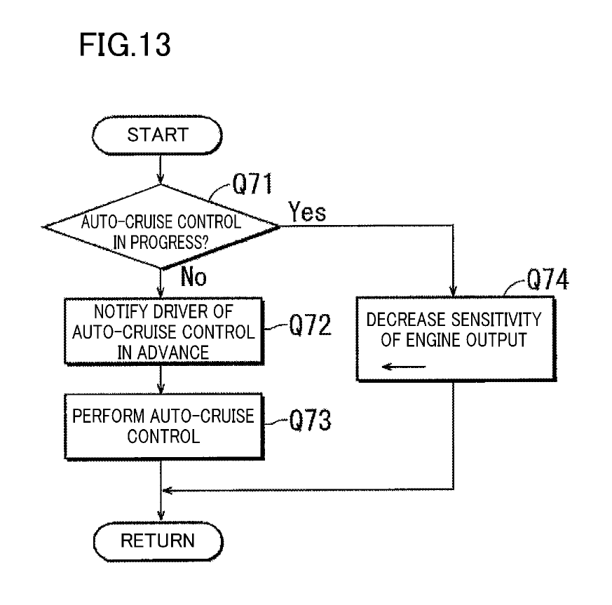

FIG. 13 illustrates an exemplary control procedure in Q8 shown in FIG. 4, i.e., an exemplary control procedure for lightening the driving operation load. Specifically, in Q71, the controller U determines whether or not auto-cruise control is being performed. If the answer to the question of this processing step Q71 is NO, the controller U notifies in Q72 the driver in advance of the auto-cruise control (e.g., by emitting an audio alert or by posting text announcement on a head-up display). Thereafter, in Q73, the auto-cruise control starts to be performed. If the answer to the question of the processing step Q71 is NO, then a control procedure of decreasing the sensitivity of the engine output is performed in Q74 (e.g., by selecting the throttle characteristic indicated by the one-dot-chain in FIG. 12). Optionally, automatic steering control may also be performed either instead of, or in addition to, the auto-cruise control described above. That is to say, performing automatic steering control to keep the currently selected lane may lighten the driver's driving operation load.

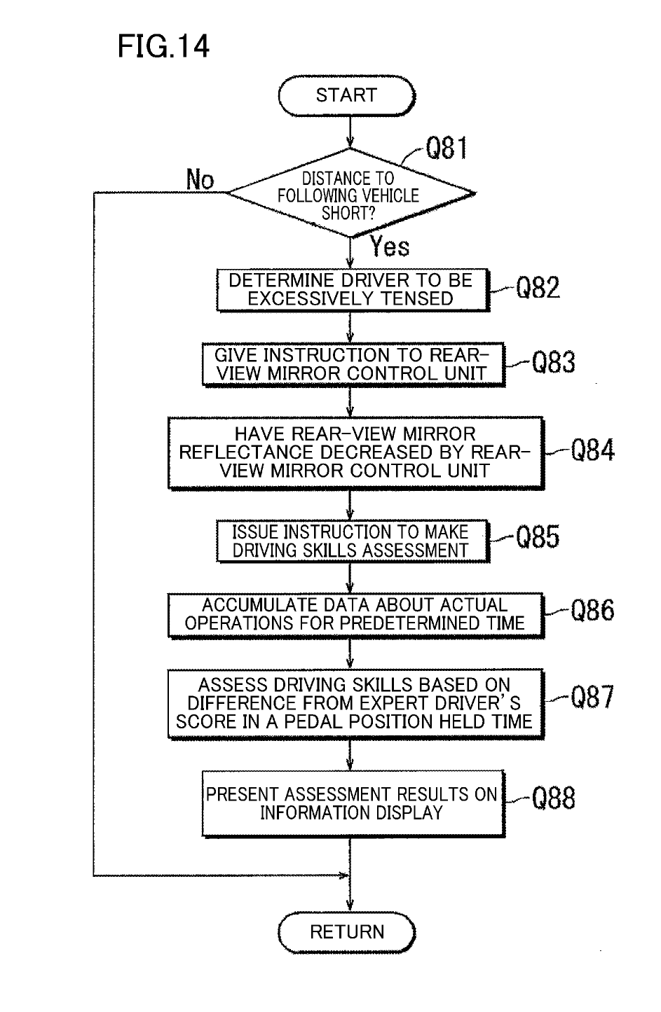

FIG. 14 illustrates an exemplary control procedure to be performed in Q7 shown in FIG. 4, i.e., an exemplary control procedure of reducing the driver's cognitive level of external factors. In this exemplary embodiment, the driver is supposed to be tensed by the following vehicle that has drawn nearer to his or her vehicle. In this embodiment, the driver's cognitive level of the following vehicle is reduced with the reflectance of the rear-view mirror S16 decreased.

Specifically, first, in Q81, the controller U determines whether or not the distance to the following vehicle is short (i.e., whether or not the distance is equal to or smaller than a threshold value that has been determined in advance according to the vehicle's speed). If the answer to the question of this processing step Q81 is NO, then the process returns with the rest of the control procedure skipped. On the other hand, if the answer to the question of this processing step Q81 is YES, then the controller U finds the driver excessively tensed in Q82. After that, in Q83, the controller U instructs the rear-view mirror control unit to decrease the reflectance of the rear-view mirror S16 to a value lower than a normal reflectance in Q84, thereby reducing the driver's cognitive level of the following vehicle. Thereafter, the controller U performs the processing steps Q85-Q87 to make the driving skills assessment, which correspond to the series of control processing steps Q51-Q54 shown in FIG. 8 and will not be described all over again to avoid redundancies. Then, in Q88, the result of assessment may be displayed on the head-up display S11, for example (i.e., helpful information for improvement of driving skills is displayed). Optionally, to reduce the driver's cognitive level of the following vehicle, the image of the following vehicle on the rear-view mirror S16 may be displayed in a smaller size than normal either instead of, or in addition to, the decreased reflectance.

The control procedure shown in FIG. 14 is only an exemplary one. Alternatively, the driver's cognitive level of external factors may also be reduced with the display modes of side view mirrors of an LCD type (not shown), for example, changed. Specifically, when the driver is drawing his or her vehicle nearer to, or away from, something (e.g., drawing it nearer to a wall), an obstacle (e.g., a fixed object or another vehicle) located diagonally behind his or her vehicle may get him or her tensed. In that case, the driver's cognitive level (sense of closeness) of the obstacle may be reduced with the image of the lateral obstacle on the side view mirror displayed in a smaller area than normal (in particular, with the image of the obstacle displayed at a position closer to the outer edge of the mirror in the vehicle width direction than normal).

Although some embodiments of the present disclosure have been described, they are just non-limiting exemplary embodiments of the present disclosure. Thus, those exemplary embodiments may be readily modified appropriately without departing from the scope of the present disclosure defined by the appended claims.

For example, the conditions for providing the driving assistance to increase the driver's internal focus on driving in Q6 shown in FIG. 4 may be any one condition or two selected from the group consisting of: whether the driver's physical condition is good or bad, whether or not there is any traffic jam, and whether the driver's driving skills are high or low. Alternatively, the driving assistance may also be provided under any other condition. Still alternatively, the driving assistance to increase the driver's internal focus on driving may also be provided whenever the driver has been found to be tensed by being forced to increase his or her external focus on driving. A specific technique for providing the driving assistance to increase the driver's internal focus on driving may be not only any of the ones described in the foregoing description of embodiments, but also any other appropriate one such as increasing the sensitivity of the steering wheel. Furthermore, the parameters used to classify the driver's condition as any of the four types shown in FIG. 2 may be only some of the ones shown in FIG. 3 or may further include any other appropriate parameter such as a one indicating a steering operation state. Optionally, an engine sound (which may also be a fake engine sound) may be output from the loudspeaker S12. In that case, the engine sound output may be amplified in Q6 shown in FIG. 4, but may be weakened in Q7. Furthermore, each of the processing steps or each series of processing steps shown in the various flowcharts represents any of the functions of the controller U. Thus, any of these functions may also be embodied as a hardware component that forms part of the controller U if the step or the series of steps is implemented as a means or section for performing its/their intended function. Naturally, objects of the present disclosure include not only the explicitly specified ones but also others that are implicitly suggested herein as advantages or benefits of the present disclosure.

What is claimed is:

1. A driving assistance system comprising:
a tension gauging unit configured to determine whether or not a driver is tensed by being forced to drive his or her vehicle passively and highly increase his or her external focus on driving; and
a driving assistance unit configured to provide, if the tension gauging unit has found the driver tensed, driving assistance that enhances the driver's motivation to drive by encouraging him or her to drive actively and thereby increase his or her internal focus on driving.

2. The driving assistance system of claim 1, further comprising:
a driver's condition detecting unit configured to detect the driver's condition;
a vehicle operating state detecting unit configured to detect the driver's vehicle operating state; and
an equipment operating state detecting unit configured to detect the driver's operating state of onboard equipment irrelevant to his or her vehicle driving operations, wherein
the system determines, based on results of detection obtained by the respective detecting units, the driver's degree of focus on driving and his or her degree of leeway in driving, and
the system finds the driver tensed if his or her degree of focus on driving is high, his or her degree of leeway in driving is low, and his or her external focus on driving has been increased by some external factors.

3. The driving assistance system of claim 2, further comprising
a driving skills assessment unit configured to assess the driver's driving skills, wherein
if the driving skills assessment unit has determined his or her driving skills to be high, the driving assistance unit provides the driving assistance that enhances the driver's motivation to drive by encouraging him or her to increase his or her internal focus on driving.

4. The driving assistance system of claim 2, further comprising:
a physical condition detecting unit configured to detect the driver's physical condition; and
a traffic jam detecting unit configured to detect any traffic jam, wherein
if the physical condition detecting unit has found the driver in good health and the traffic jam detecting unit has detected no traffic jams, the driving assistance unit provides the driving assistance that enhances the driver's motivation to drive by encouraging him or her to increase his or her internal focus on driving.

5. The driving assistance system of claim 2, further comprising
a classification unit configured to classify, based on results of detection obtained by the respective detecting units, the driver's condition as any of a plurality of types using, as parameters, his or her degree of focus on driving and his or her degree of leeway in driving, wherein
the classification unit classifies the driver's condition as:
a first type if his or her degree of focus on driving is low, his or her degree of leeway in driving is low, and his or her degree of distraction from driving is high;
a second type if his or her degree of focus on driving is low and his or her degree of leeway in driving is high;
a third type if his or her degree of focus on driving is high, his or her degree of leeway in driving is low, and his or her degree of external focus on driving has been increased by some external factors; or
a fourth type if his or her degree of focus on driving is high, his or her degree of leeway in driving is high, and his or her degree of internal focus on driving is high, and
if the classification unit classifies the driver's condition as the third type, the tension gauging unit finds the driver tensed.

6. The driving assistance system of claim 5, further comprising:
a degree of internal focus measuring unit configured to measure the degree of internal focus of the driver who is driving actively;
a degree of external focus measuring unit configured to measure the degree of external focus of the driver who is driving passively;
a degree of distraction measuring unit configured to measure the driver's degree of distraction from driving operations; and
a degree of spare capacity measuring unit configured to measure the degree of spare capacity indicating the driver's degree of loss of focus, wherein
the classification unit classifies, based on results of detection obtained by these four measuring units, the driver's condition as any of the various types.

7. The driving assistance system of claim 6, wherein
the classification unit classifies the driver's condition as any of the various types by regarding the degree of internal focus and the degree of spare capacity as values indicating the driver's degree of leeway and also regarding the degrees of internal and external foci as values indicating the driver's degrees of focus.

8. The driving assistance system of claim 2, wherein
the driver's condition detecting unit includes an image capturing unit configured to capture an image covering at least the driver's face, and
the vehicle operating state detecting unit includes at least two sensors configured to detect respective operating states of an accelerator pedal and a brake pedal.

9. The driving assistance system of claim 1, wherein
the driving assistance includes giving exemplary driving instructions to the driver.

10. The driving assistance system of claim 1, wherein
the driving assistance includes providing the driver with navigation to a road with features that would entertain him or her through driving.

11. The driving assistance system of claim 1, wherein
the driving assistance includes improving sensitivity to any change in the vehicle's state responsive to driving operations.

12. A driving assistance system comprising:
a tension gauging unit configured to determine whether or not a driver is tensed by being forced to drive his or her vehicle passively and highly increase his or her external focus on driving;
a driving assistance unit configured to provide, if the tension gauging unit has found the driver tensed, driving assistance that enhances the driver's motivation to drive by encouraging him or her to drive actively and thereby increase his or her internal focus on driving; and
a driving skills assessment unit configured to assess the driver's driving skills, wherein
if the driving skills assessment unit has determined his or her driving skills to be high, the driving assistance unit provides the driving assistance that enhances the driver's motivation to drive by encouraging him or her to increase his or her internal focus on driving.

13. The driving assistance system of claim 12, wherein
the driving assistance includes giving exemplary driving instructions to the driver.

14. The driving assistance system of claim 12, wherein
the driving assistance includes providing the driver with navigation to a road with features that would entertain him or her through driving.

15. The driving assistance system of claim 12, wherein
the driving assistance includes improving sensitivity to any change in the vehicle's state responsive to driving operations.

16. A driving assistance system comprising:
a tension gauging unit configured to determine whether or not a driver is tensed by being forced to drive his or her vehicle passively and highly increase his or her external focus on driving;
a driving assistance unit configured to provide, if the tension gauging unit has found the driver tensed, driving assistance that enhances the driver's motivation to drive by encouraging him or her to drive actively and thereby increase his or her internal focus on driving;
a physical condition detecting unit configured to detect the driver's physical condition; and
a traffic jam detecting unit configured to detect any traffic jam, wherein
if the physical condition detecting unit has found the driver in good health and the traffic jam detecting unit has detected no traffic jams, the driving assistance unit provides the driving assistance that enhances the driver's motivation to drive by encouraging him or her to increase his or her internal focus on driving.

17. The driving assistance system of claim 16, wherein
the driving assistance includes giving exemplary driving instructions to the driver.

18. The driving assistance system of claim 16, wherein
the driving assistance includes providing the driver with navigation to a road with features that would entertain him or her through driving.

19. The driving assistance system of claim 16, wherein
the driving assistance includes improving sensitivity to any change in the vehicle's state responsive to driving operations.
